(19) United States
(12) Patent Application Publication
(10) Pub. No.: US 2006/0263458 A1
(43) Pub. Date: Nov. 23, 2006

(54) Composition comprising growth factor of xanthoceras sorbifolia extracts, compounds isolated from same, methods for preparing same and uses thereof

(76) Inventors: May Sung Mak, Kowloon (HK); Pui-Kwong Chan, Sugarland, TX (US)

Correspondence Address: Albert Wai-Kit Chan;Law Offices of Albert Wai-Kit Chan, LLC, World Plaza, Suite 604, 141-07 20th Avenue, Whitestone, NY, 11357, US

(21) Appl. No.: 11/412,659

(22) Filed: Apr. 27, 2006

Related U.S. Application Data
(63) Continuation-in-part of application No. 11/289,142, filed on Nov. 28, 2005.
(63) Continuation-in-part of application No. 11/267,523, filed on Nov. 4, 2005.
(63) Continuation-in-part of application No. PCT/US05/31900, filed on Sep. 7, 2005.
(63) Continuation-in-part of application No. 11/131,551, filed on May 17, 2005.
(63) Continuation-in-part of application No. 11/117,760, filed on Apr. 27, 2005.
(63) Continuation-in-part of application No. 10/906,303, filed on Feb. 14, 2005.
Application No. 10/906,303 is a continuation-in-part of application No. PCT/US04/43465, filed on Dec. 23, 2004.
Application No. PCT/US04/43465 is a continuation-in-part of application No. PCT/US04/33359, filed on Oct. 8, 2004.
(60) Provisional application No. 60/675,282, filed on Apr. 27, 2005.
(60) Provisional application No. 60/675,284, filed on Apr. 27, 2005.
(60) Provisional application No. 60/532,101, filed on Dec. 23, 2003.
(60) Provisional application No. 60/509,851, filed on Oct. 9, 2003.
(60) Provisional application No. 60/617,379, filed on Oct. 8, 2004.
(60) Provisional application No. 60/613,811, filed on Sep. 27, 2004.
(60) Provisional application No. 60/607,858, filed on Sep. 7, 2004.

(51) Int. Cl. A61K 36/77

(52) U.S. Cl. 424/769

(57) ABSTRACT

This invention provides a process of producing extract comprises growth-stimulation components from husks, kernels, leaves, fruit-stems, branches, stems, roots, seed's shells and barks and their applications. A use of composition comprising the extract from the husks, kernels, leaves, fruit-stems, branches, stems, roots, seed's shells and barks of Wenguanguo for stimulating the cell aactivities comprising growth-stimulation by administering an appropriate amount of the above composition.

This application is a Continuation-In-Part of U.S. Ser. No. 11/289,142, filed Nov. 28, 2005, Continuation-In-Part of U.S. Ser. No. 11/267,523, filed Nov. 4, 2005, Continuation-In-Part of International Application No. PCT/US05/31900, filed Sep. 7, 2005, Continuation-In-Part of U.S. Ser. No. 11/131,551, filed May 17, 2005, Continuation-In-Part of U.S. Ser. No. 11/117,760, filed Apr. 27, 2005, Continuation-In-Part of U.S. Ser. No. 60/675,282, Filed Apr. 27, 2005, Continuation-in-Part of U.S. Ser. No. 60/675,284, Filed Apr. 27, 2005. Continuation-In-Part of U.S. Ser. No. 10/906,303, filed Feb. 14, 2005 which is a Continuation-In-Part of International Application No. PCT/US04/43465, filed Dec. 23, 2004, which is a Continuation-In-Part of International Application No. PCT/US04/33359, filed Oct. 8, 2004, which claims the benefit of U.S. Ser. Nos. 60/532,101, filed Dec. 23, 2003, and 60/509,851, filed Oct. 9, 2003; and International Application No. PCT/US05/31900, filed Sep. 7, 2005, claims the benefit of U.S. Ser. Nos. 60/617,379, filed Oct. 8, 2004, 60/613,811, filed Sep. 27, 2004, and 60/607,858, filed Sep. 7, 2004. The contents of these preceding applications are hereby incorporated in their entireties by reference into this application.

Throughout this application, various publications are referenced. Disclosures of these publications in their entireties are hereby incorporated by reference into this application in order to more fully describe the state of the art to which this invention pertains.

### FIELD OF THE INVENTION

This invention relates to extracts from a plant called Wenguanguo (Xanthoceras Sorbifolia), their usages and functions, and methods of their preparation

### BACKGROUND OF THE INVENTION

Wenguanguo is a species of the sapindaceae family. Its scientific name is Xanthoceras sorbifolia Bunge. Wenguanguo is the common Chinese name; others are Wenguannguo, Wenguanmu, Wenguanhua, Xilacedeng, Goldenhorn and Yellowhorn. Wenguanguo is grown in Liaoning, Jilin, Hebei, Shandong, Jiangsu, Henan, Shanxi, Shaanxi, Gansu, Ningxia and Inner Mongolia, China. Its seeds, leaves and flowers are edible and have been used as a folk medicine to treat enuresis for centuries. Its branches and woods are also used as a folk medicine.

Chinese patent applications CN 1092991A and CN 1092992A provided the methods for producing a medicine from Wenguanguo kernel powder for curing enuresis and enhancing cerebral functions. Chinese patent CN 1052636C provided a method for producing a medicine with ethanol extract from the Wenguanguo kernel powder for curing enuresis and enhancing cerebral functions. Journal of Shenyang University of Pharmacy (2001), 18(1), 53-56 disclosed the n-butanol extract from the wood of Wenguanguo, which has anti-inflammatory effect.

United States Patent Application Publication No. 20030096030 provided the extracts from the husks of Wenguanguo which are Bunkankasaponin A. B. C. D and two sterols for preventing cerebral aging, improving cerebral functions and curing enuresis, frequent micturition, urinary incontinence, dementia, weak intelligence, and increasing the body's ability to resist the activity of glycosuria.

United States Patent Application Publication No. 20030082293 disclosed the extracts Bunkankasaponin A. B. C. D, crude fats, crude protein and sugars from the shell of Wenguanguo.

U.S. Pat. No. 6,616,943, issued on Sep. 9, 2003, provides the composition comprising Wenguanguo combined extracts and the methods for preparing them and uses thereof. The methods for preparing the combined extract from the husks comprise the following steps: extracting Wenguanguo husks with an organic solvent (e.g. ethanol) to form an organic extract; removing the organic solvent from the organic extract to form aqueous extracts; drying and sterilizing the aqueous extracts to form the combined extracts. The combined extracts contain saponins, saccharides, proteins and others. The extracts can be used for producing medicines or health foods for preventing cerebral aging, improving memory, improving cerebral functions and curing enuresis, frequent micturition, urinary incontinence, dementia, weak intelligence and Alzheimer's disease, autism, brain trauma, Parkinson's disease and other diseases caused by cerebral dysfunction. The medicines or health foods further comprise Vitamin B, Vitamin D, K, anti-oxidant, Cordyceps or its extracts, gingko or its extracts, Echinacea, or its extracts, Huperzine A, folic acid, amino acids, creatine, fiber supplement or a combination thereof.

Yingjie Chen, Tadahiro Takeda and Yukio Ogihara in Chem. Pharm. Bull 33(4)1387-1394(1985) describe a study on the constituent of Xanthoceras sorbifolia Bunge. See Section V. Saponins from the Fruits of Xanthoceras sorbifolia. Four new saponins were isolated from the fruits of Xanthoceras sorbifolia Bunge. The structures of these saponins are bunkankasaponins A, B, C and D:

Yingjie Chen, Tadahiro Takeda and Yukio Ogihara in Chem. Pharm. Bull 33(3)1043-1048(1985) describe studies on the constituent of Xanthoceras sorbifolia Bunge. See Section IV. Structures of the Miner Prosapogenin. The prosapogenins from the partial hydrilyzate of fruit saponin of Xanthoceras sorbifolia were examinated, and are characterized as:
16-O-acetyl-21-O-(3,4-di-O-angeloyl-β-D-fucopyranosyl) protoaecigenin
22-O-acetyl-21-O-(3,4-di-O-angeloyl-β-D-fucopyranosyl) protoaecigenin 3-O— β-D-glucuronopyranoside.

Yingjie Chen, Tadahiro Takeda and Yukio Ogihara in Chem. Pharm. Bull 33(1)127-134(1985) describe studies on the constituent of Xanthoceras sorbifolia Bunge. See Section III. Minor Prosapogenins aponins from the Fruits of Xanthoceras sorbifolia Bunge. The structure of 3 minor prosapogenins, obtained by acid hydrolysis of the crude saponin faction, were characterized as:
21-O-(3,4-di-O-angeloyl)-β-D-fucopyranosyltheasapogenol B
21-O-(4-O-acetyl-3-O-angeloyl)-β-D-fucopyranosyltheasapogenol B
21-O-(4-O-acetyl-3-O-angeloyl)-β-D-fucopyranosyl-22-O-acetylprotoaescigenin

Yingjie Chen, Tadahiro Takeda and Yukio Ogihara in Chem. Pharm. Bull 33(4)1387-1394(1985) described a study on the constituent of Xanthoceras sorbifolia Bunge. See Section II. Major Sapogenol and prosapogenin from the Fruits of Xanthoceras sorbifolia.

### SUMMARY OF THE INVENTION

In accordance with these and other objects of the invention, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

This invention is related to the use of extracts of Wenguanguo. The extracts of Wenguanguo can prevent patients from enuresis, bedwetting, incontinence and frequency micturition. The extracts of Wenguanguo prevent enuresis because it improves patients' urinary tract functions.

This invention provides a composition to improve the signal pathway between bladder and central nervous system to help patients be more aware of the signal sent from the bladder and wake themselves from deep sleep.

This invention provides a composition which strengthens the bladder so that it can store more urine.

The extracts of Wenguanguo may be used to strengthen the detrusor and sphincter function which is dyfunction caused by aging, stress, nervousness, over-activity, instability, hyper-reflexia, and uninhibited bladder. The extracts of Wenguanguo may be used as acetylcolinesterase (AChE) inhibitor, and/or regulating Antidiuretic hormone (ADH), which then reduces the volume of urine. This invention provides a process of producing extract comprises growth-stimulation components from husks, kernels, leaves, fruit-stems, branches, stems, roots, seed's shells and barks and their applications. This invention provides a composition comprising the extract from the husks, kernels, leaves, fruit-stems, branches, stems, roots, seed's shells and barks of Wenguanguo. The extract comprises growth-stimulation components.

Wenguanguo extracts may be used for accelerating the growth of bladder, for suppressing deep sleep, for increasing alertness in a sleeping subject, for modulating the release, breakdown and uptake of Antidieuretic hormone (ADH) and its receptors, for modulating the secretion, breakdown and uptake of Adrenocorticotropic hormone (ACTH) and its receptors, for modulating the release, breakdown and uptake of 5-hydroxytryptamine and its receptors, for modulating the release, breakdown and uptake of Acetycholine (Ach) and its receptors, for modulating the release, breakdown and uptake of Adrenaline (AD) and its receptors, for modulating the release, breakdown and uptake of Dopamine (DA) and its receptors, for modulating the release, breakdown and uptake of Norepinephrine (NE) and its receptors, for preventing sleep paralysis, for modulating the formation, release, breakdown and activity of neuropeptides and their receptors and for improving the functions of bladder.

This invention provides a method for isolating compounds from Xanthoceras Sorbifolia comprising steps of: extracting Xanthoceras Sorbifolia powder with an appropriate amount of one or more organic solvents for an appropriate amount of time to form an organic extract; collecting the organic extract; refluxing the organic extract with an organic solvent for an appropriate amount of time to form a second extract; removing the organic solvent from the second extract; and drying and sterilizing the second extract to form a Xanthoceras Sorbifolia extract powder.

This invention provides a method for isolating compounds from Xanthoceras Sorbifolia comprising steps of: extracting Xanthoceras Sorbifolia powder with an appropriate amount of one or more organic solvents for an appropriate amount of time to form an organic extract; collecting the organic extract; refluxing the organic extract to form a second extract; removing the organic solvent from the second extract; drying and sterilizing the second extract to form a Xanthoceras Sorbifolia extract powder; fractionating the extract powder to obtain one or more components of the extract powder; identifying the bioactive components of the extract powder; purifying one or more bioactive components of the extract powder with chromatographic methods with FPLC technology to obtain one or more bioactive components; and final isolating the compound with preparative HPLC.

The compounds can be isolated from the plant called Xanthoceras Sorbifolia or can be synthesized chemically, or extracted from other biological sources.

The Xanthoceras Sorbifolia derived compound and/or composition regulates the cell's components or receptors and strengthen the cell growth process.

The Xanthoceras Sorbifolia derived compound and/or composition regulates the cell's enzymes activitieIn accordance with these and other objects of the invention, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

### DETAILED DESCRIPTION OF THE FIGURES

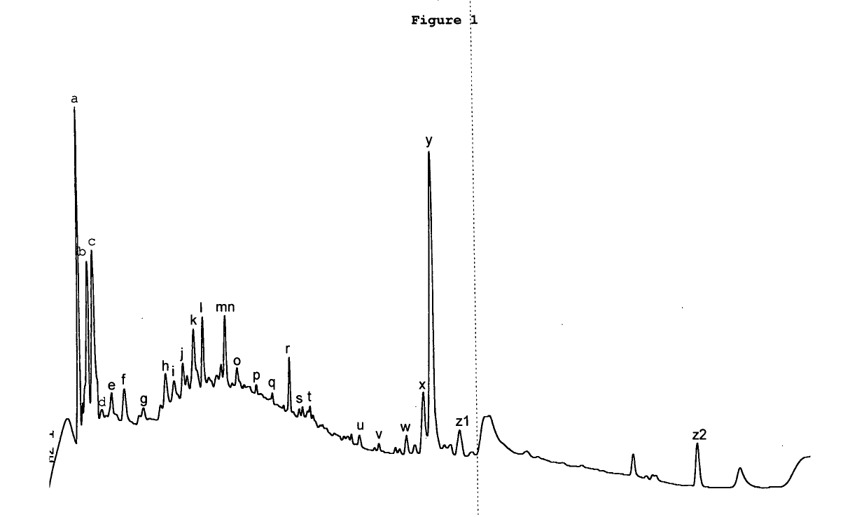

FIG. 1 shows the HPLC Profile of Wanguanguo husk extract.

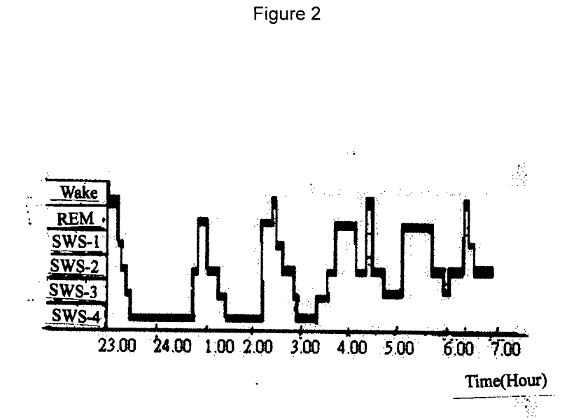

FIG. 2 shows the sleep cycle of a typical person.

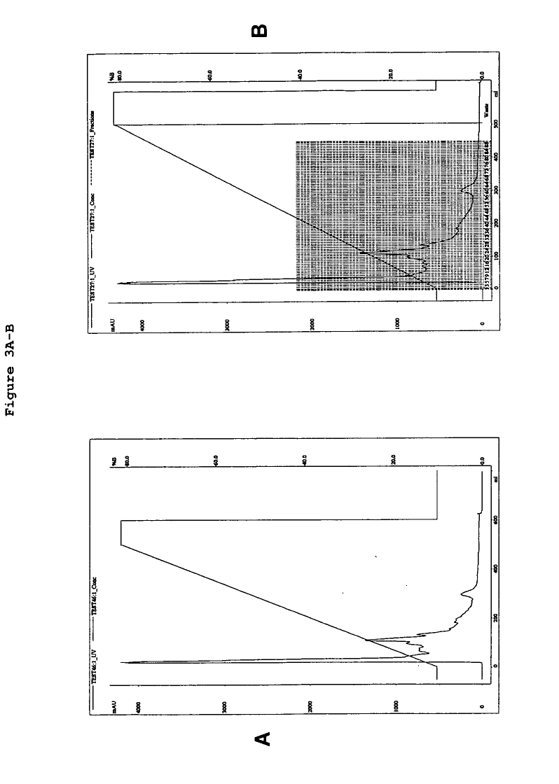

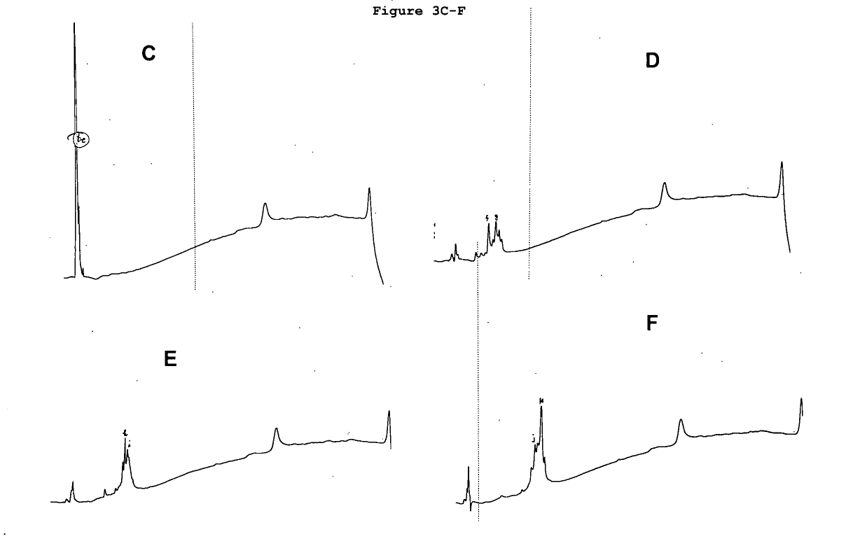

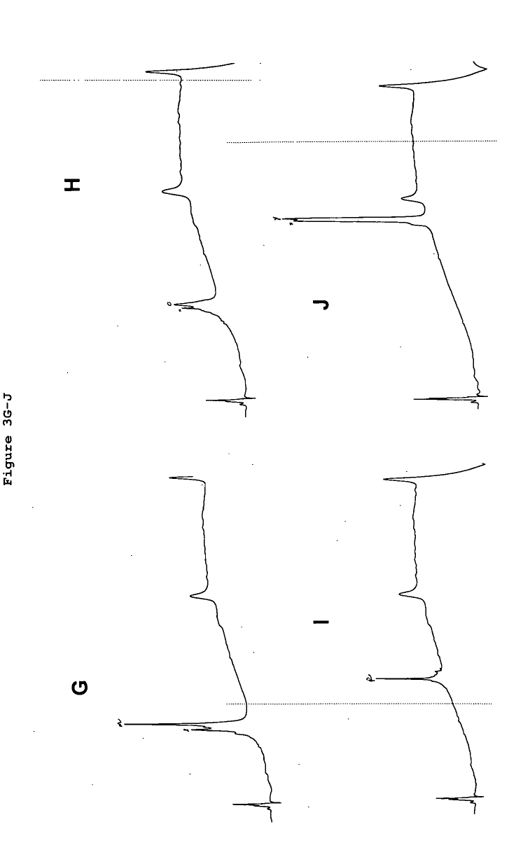

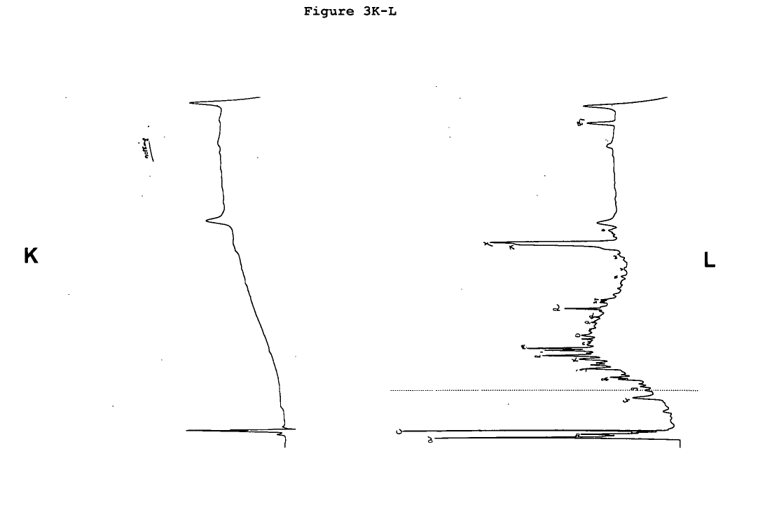

FIGS. 3A-L shows the identification of fractions of the Xanthoceras Sorbifolia extract by FPLC and HPLC. A and B are representative elution profiles of FPLC. C to K are HPLC profiles of fractions obtained from the FPLC. L: original materials.

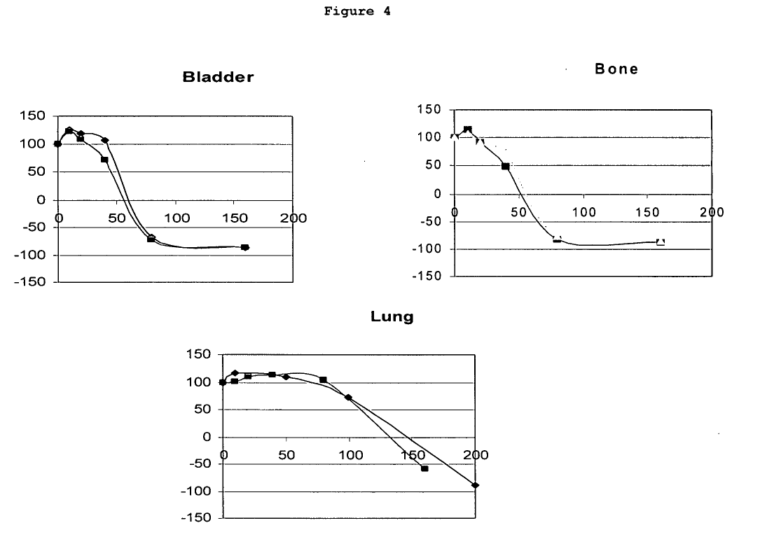

FIG. 4 shows the efficacy of the Wanguanguo extracts in bladder, lung and bone cells. Experimental results showed that these cells began to grow at low concentration of 10 ug/ml. In lung cells, it grew up to 120% in extract concentration of 50 ug/ml. The bladder cells were stimulated to grow up to 125% in culture medium RPMI-1640 containing extract concentration of 10 μg/ml. The results also show the efficacy of Wanguanguo extract on the activity of bone cells' growth. The extract stimulates its growth up to 120% of controls when used with low concentration of extract (i.e., 10 μg/ml).

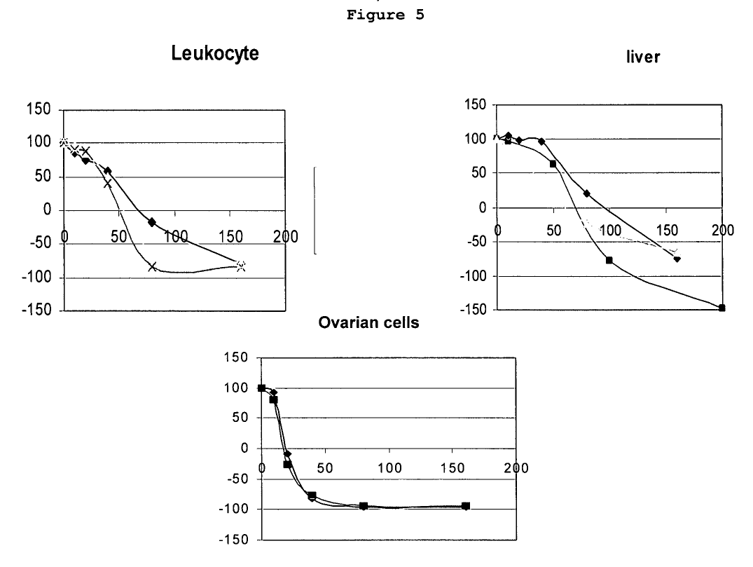

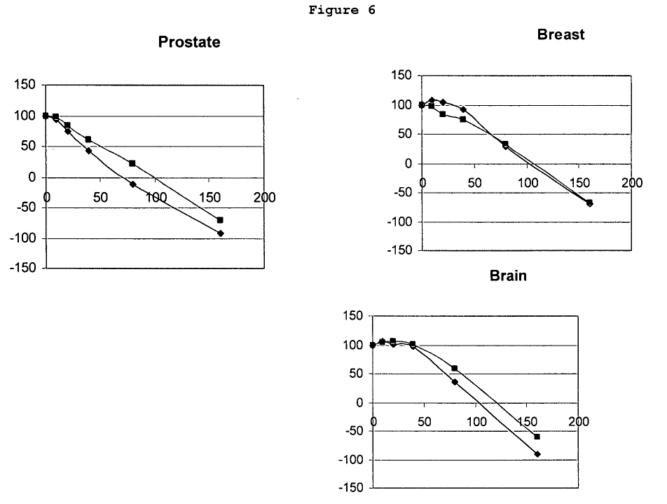

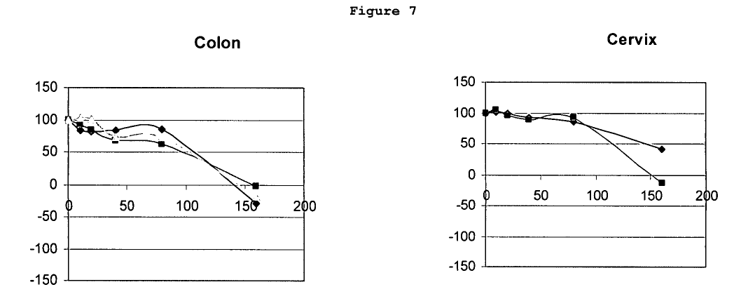

FIGS. 5-7 show the growth curves of cells after treatment with the extract of Xanthoceras Sorbifolia as determined by MTT assay. These studies indicate there are differences in sensitivity in these cell lines toward the extract of Xanthoceras Sorbifolia.

FIG. 8 shows the elution profile of an extract of Xanthoceras Sorbifolia in FPLC with 10-80% acetonitrile gradient. Ordinate: Optical Density at 245 nm (mAU). Abscissa: volume of elution (ml).

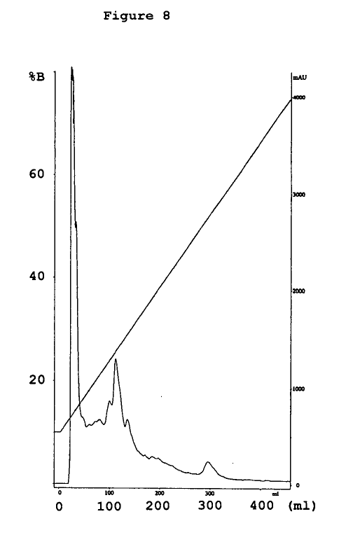

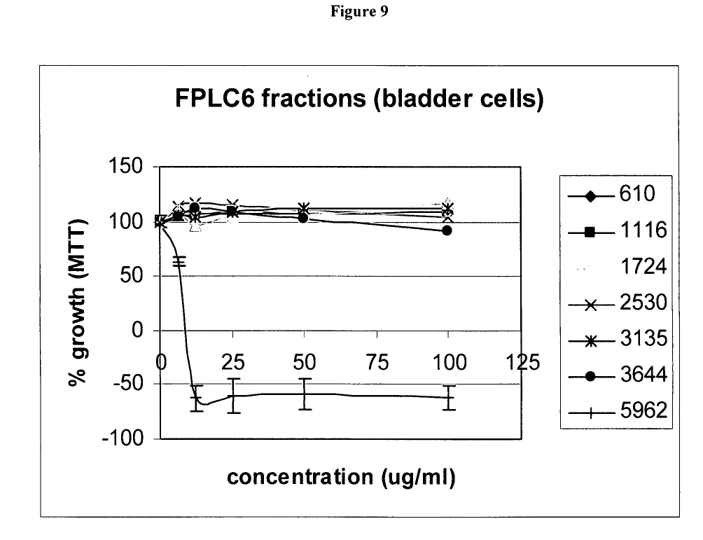

FIG. 9 shows screening of cell growth component from the FPLC fractions (conducted with bladder cells). Fractions obtained from FPLC (FIG. 8) were pooled and used in MTT assay. This figure shows the cell-growth activity induced by different components of Xanthoceras Sorbifolia extracts. Both growth and inhibition effects on cells were found. Fractions 1116, 1724 and 2530 have small stimulation activity of cell growth. Abscissa: concentration (ug/ml)

Ordinate: % Cell Growth (determined by MTT assay).

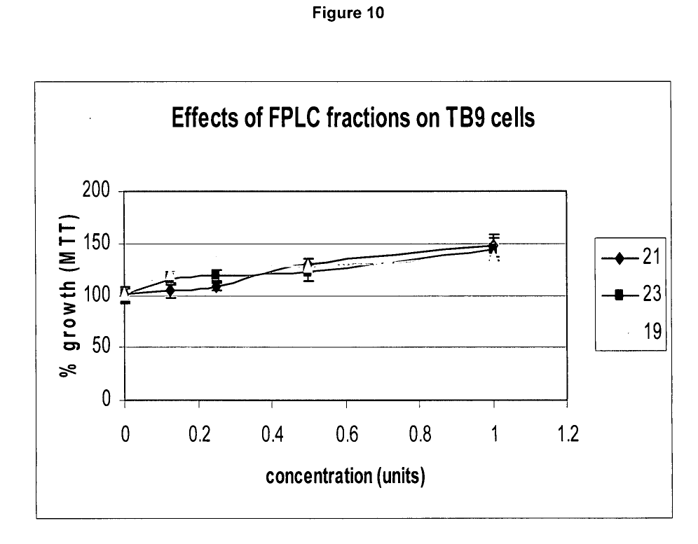

FIG. 10 shows stimulation activity of FPLC fractions 19, 21 and 23 on the growth of TB9 cells.

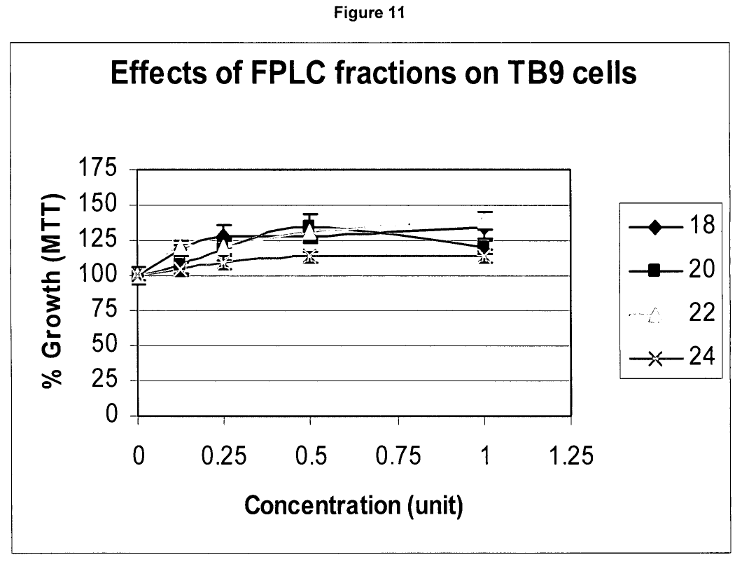

FIG. 11 shows the stimulation activity of FPLC fractions 18, 20, 22 and 24 on the growth of TB9 cells.

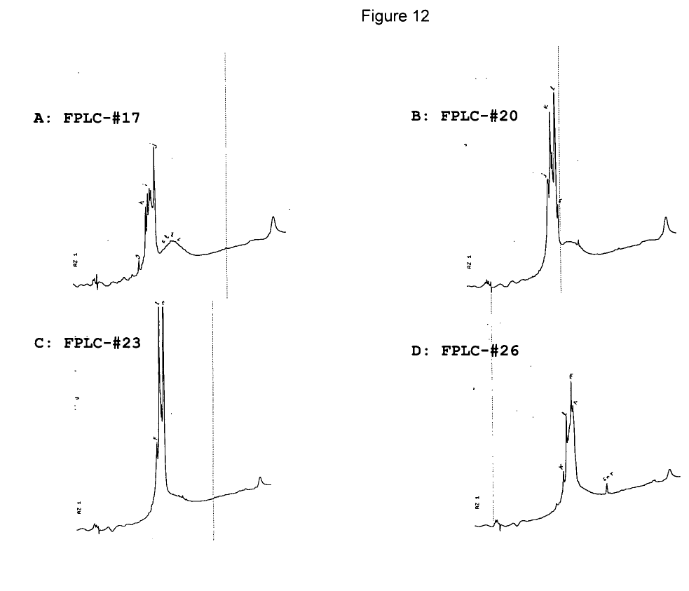

FIG. 12 A-D show the HPLC elution profiles of the FPLC fractions 17, 20, 23 and 26. A C18 reverse phase column was employed. The content was eluted with a 10-80% acetonitrile gradient. 5-6 major components are identified (for example, in fraction #20).

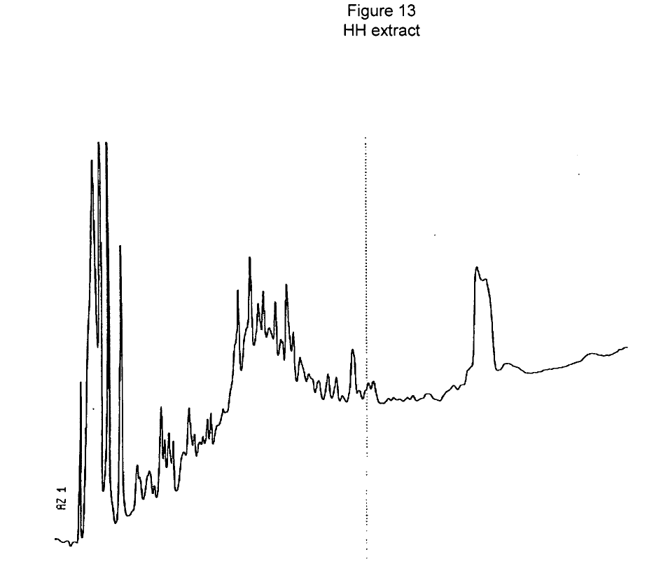

FIG. 13 shows the HPLC Profile of Wanguanguo HH extract.

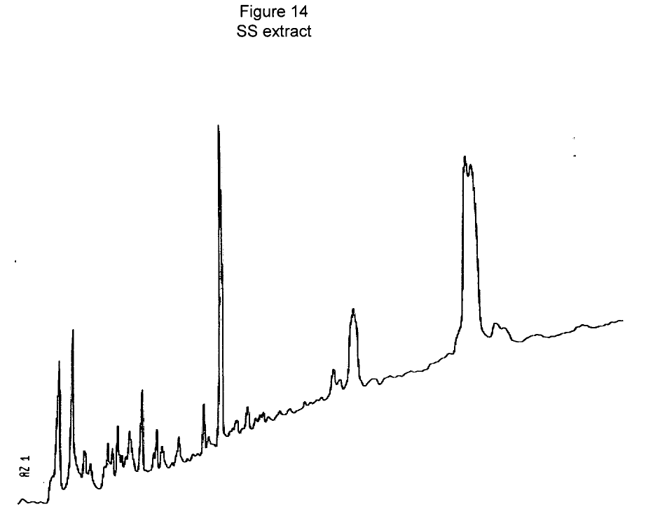

FIG. 14 shows the HPLC Profile of Wanguanguo SS extract.

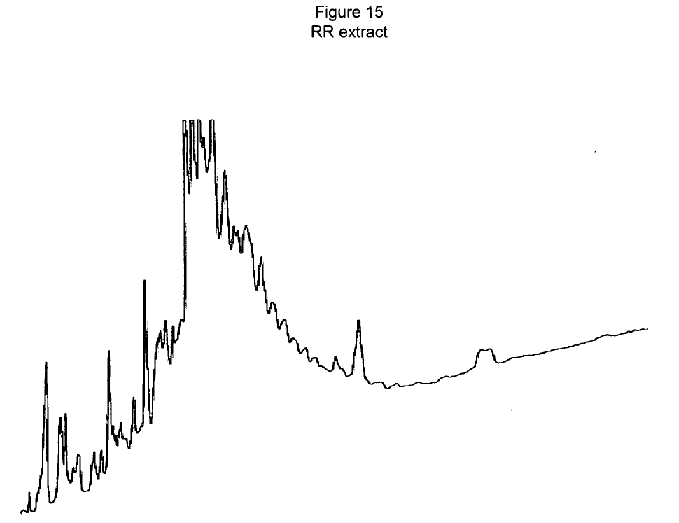

FIG. 15 shows the HPLC Profile of Wanguanguo RR extract.

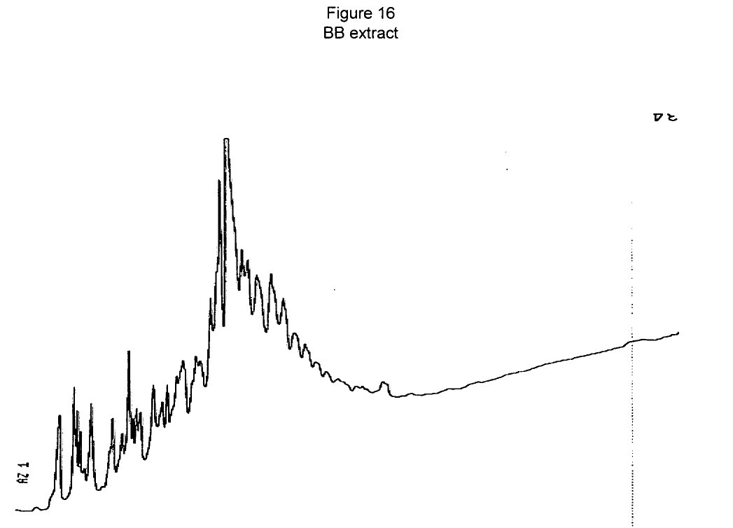

FIG. 16 shows the HPLC Profile of Wanguanguo BB extract.

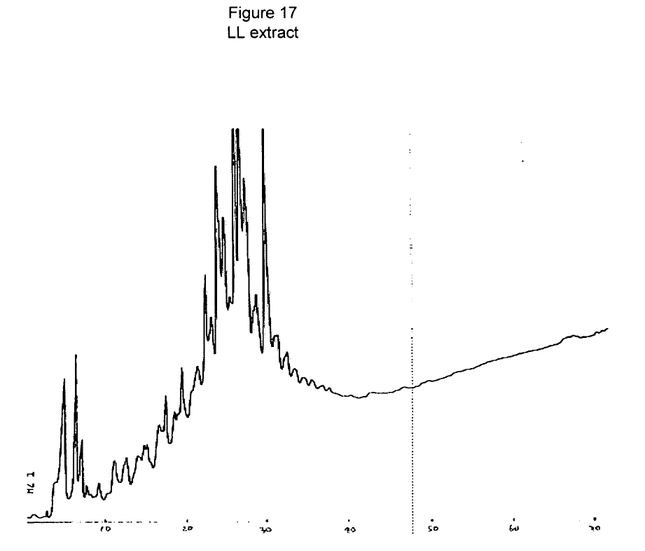

FIG. 17 shows the HPLC Profile of Wanguanguo LL extract.

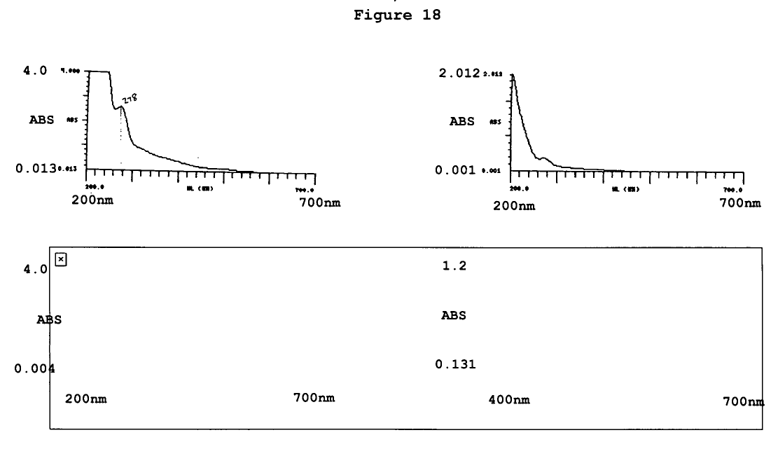

FIG. 18 shows the absorption spectrum of Xanthoceras Sorbifolia extract. Abscissa: Wavelength in nm. Ordinate: Optical Density. The extract has three absorption maximum at 207 nm, 278 nm and 500 nm.

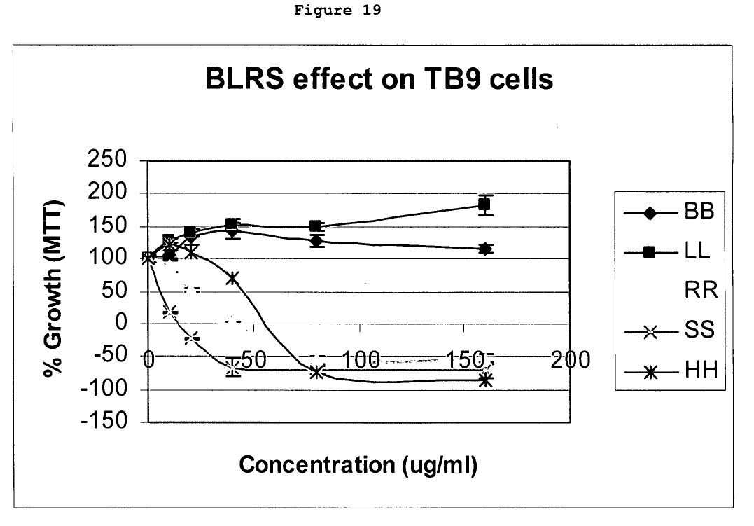

FIG. 19 shows the growth curves of bladder cells treated with extracts from different parts of plant. The SS, RR and HH have inhibition activities but the LL and BB extract have stimulation activities indicate containing stimulation component(s).

### DETAILED DESCRIPTION OF THE INVENTION

According to the theory of traditional Chinese medicine, enuresis, frequent micturition and urinary incontinence are caused by “deficiency in kidney (“shen”)”. Therefore, they are treated by using Chinese herbs which can tone the kidney, such as Ginseng Bajitian, Roucongrong Duzhong and Cordyceps. These herbs can strengthen function of the kidney and regulate water metabolism of human's body through the “kidney pathway” that will help with curing the enuresis, frequent micturition and urinary incontinence.

It is hypothesized that the Wenguanguo extracts cure the enuresis, frequent micturition and urinary incontinence through the “bladder pathway” to regulate water metabolism of human's body and urination. The Wenguanguo extracts of the present invention stimulate the growth of the bladder. The Wenguanguo extracts of the present invention increase the capacity of bladder and function of bladder controlling the urination. In another aspect of the present invention, Wenguanguo extracts, when used with the “kidney pathway” herbs to treat the enuresis, frequent micturition and urinary incontinence, will strengthen both the pathways of kidney and bladder, and then will produce better results on treatments.

Wenguanguo is a species of the sapindaceae family. Its scientific name is Xanthoceras sorbifolia Bunge. Wenguanguo is the common Chinese name; others are Wenguannguo, Wenguanmu, Wenguanhua, and Xilacedeng. This plant can grow up to 8 meters in height. It features odd pinnately compound leaf, eraceme with white flowers, capsules with thick and woody husks. Wenguanguo is grown in Liaoning, Jilin, Hebei, Shandong, Jiangsu, Henan, Shanxi, Shaanxi, Gansu, Ningxia and Inner Mongolia, China. Its seeds are edible and have been used as a folk medicine to treat enuresis for centuries. Its branches and woods are also used as a folk medicine.

This invention is a further description of the extracts from Wenguanguo, their uses and methods for preparation. This invention provides the extracts that can regulate the enzymes activities of bladder cell.

The Xanthoceras Sorbifolia derived compounds and/or composition regulates the cell's components or receptors and strengthen the cell growth process.

The Xanthoceras Sorbifolia derived compound and/or composition regulates the activities of cell's enzymes.

This invention provides the extracts that can prevent enuresis by improving patients' signal pathway from bladder to cerebral so that patients can be more aware of the signals sent from the bladder and wake up from deep sleep. When the bladder is full of urine, the smooth muscle of the bladder is extended, which produces a signal up to the cerebral cortex and cerebellum through the pelvic nerve and the sacral spinal cord. The response of the cerebral cortex and cerebellum to the signal is to make the bladder sustainably contracted but the sphincter relaxed. The urine is then discharged. When the bladder is filled with urine via the urethra during sleep, the detrusor stretches, allowing the bladder to expand. As the bladder starts to accumulate urine, it will stimulate the stretch receptors in the bladder that will generate signals continually to the brain according to the amount of urine accumulated in the bladder. When the bladder is full enough with urine, then the intravesicle has accumulated enough pressure for the brain to recognize and wake the person to urinate. If the signal is not strong enough to wake the sleeping person or blocked, or sphincter loose or detrusor-sphincter dyssynergia, enuresis occurs. This particular plant extract can cure enuresis.

The sensory stretch receptors are located within the bladder wall and help with assessing the degree of bladder fullness. This information is transmitted up to the spinal cord and then via the spinothalamic tracts to the central nervous system. The extracts of Wenguanguo strengthen the signal for central nervous system.

When the bladder becomes contracted under stress and nervousness, the capacity of the urinary bladder will be reduced and then the frequent micturition occurs. The extracts of Wenguanguo can strengthen the bladder for storing more urine.

The capacity of the urinary bladder is reduced because of aging, and this may even happen to middle-aged people. They suffer from experience of early detrusor contraction due to a sense of urgency to empty the bladder at low urine level. The extracts of Wenguanguo can help strengthen the detrusor and therefore the bladder capacity increases and urinary frequency decreases.

The bladder can not hold normal capacity volume of urine because of sphincter loose. This may even happen to middle-aged people. The sense of urgency to empty the bladder at low urine level will be happened. The extracts of Wenguanguo can help strengthen the sphincter and therefore the bladder capacity increases and urinary frequency decreases.

Patients with detrusor over-activity, detrusor instability, detrusor hyper-reflexia or uninhibited bladder have early, forceful detrusor contractions before the bladder is full. This creates urgency and frequency urinary discharge. The extract of Wenguanguo regulate the patient's detrusor. The bladder becomes stable and can store a full amount of urine.

The smooth muscle of the urinary bladder has two functions: When the bladder is relaxed, the urine is stored. When it is contracted, the urine will be discharged. The sensory stretch receptors are located within the bladder wall to assess the bladder's fullness. This information is transmitted up the spinal cord via the spinothalamic tracts to the nervous system. The brain generates inhibitory signals when detrusor relaxation is desired. But the brain generates excitatory signal when detrusor contraction is desired. The extracts of Wenguanguo can strengthen the bladder tissue by inhibiting Acetylcholinesterase, AchE. The inhibiting effect can be maintained for a long period of time. The extracts of Wenguanguo are a good AChE inhibitor that can cure the diseases caused by deficiency of Acetylcholine, ACh.

Antidiuretic hormone (ADH) is stored in the posterior pituitary gland in the brain. It is the primary regulator of body water. ADH acts on the kidneys to increase or decrease total body water. This has an effect on the volume of urine generated by the kidney. The release of ADH is controlled by the cells of osmoreceptors and baroreceptors. Osmoreceptors are the specialized cell hypothalamus. These cells sense the concentration of particles in the blood. When the concentration of particles is higher, more ADH will be released by the pituitary. This stimulates retention of water to dilute body fluids. When the concentration is lower, less ADH will be released by the pituitary. Baroreceptors are located in the right atria and great veins and carotid sinus the specialized area in the heart that sense blood volume and blood pressure. The heart will generate signals to the hypothalamus and pituitary to release more ADH when blood volume or blood pressure is low and vice versa. The extracts of Wenguanguo can regulate the release of ADH which will reduce the volume of urine produced by the body.

This invention relates to the extracts from Wenguanguo leaves, barks, branch, root, kernel, husks and fruit-stems, and methods of their preparation.

The methods for preparing the extract comprise the following steps: extracting powder of Wenguanguo leaves, barks, branch, root, kernel, husks and fruit-stems the husks or fruit-stems with an organic solvent (ethanol, methanol and others) at ratio of 1:2 for 4-5 times, 20-35 hours for each time to form an organic extract; collect and reflux the organic extract 2-3 times at 80° C. to form second extracts; resolve the second extracts in water to form an aqueous solution; extract the aqueous solution by n-butanol to form a n-butanol extracts; chromatograph the n-butanol extracts to form the extract.

This invention further describe fractionating the extract powder to obtain one or more components of the extract powder; identifying the bioactive components of the extract powder; purifying one or more bioactive components of the extract powder with FPLC to obtain one or more fraction of the bioactive component; and isolating the compound with preparative HPLC.

The compositions show particular activities when comprising different combination of the compounds. The characteristic elution profile or peaks are shown in FIG. 13, or FIG. 14, or FIG. 15, or FIG. 16 or FIG. 17. Their activities are showed in FIG. 19.

This invention provides the composition of Wenguanguo components for inhibiting the uptake of 5-hydroxytryptamine (5HT) in a subject.

5-HT controls and modulates a sleep factor that sustains and increases deep sleep. Inhibiting the uptake of 5HT will decrease deep sleep. People who spend too much time in SWS 3 and SWS 4 are unable to awaken from their sleep when their bladder is full because their sleep is too deep. This is the reason that enuresis often occurs during SWS 3 and SWS 4.

This invention provides the composition of Wenguanguo components for increasing the activity of Dopamine in a subject thereby making the central nerve system of said subject alert.

This invention provides the composition of Wenguanguo components for increasing the secretion of antidiuretic hormone (ADH) in a subject, which reduces urine in said subject.

This invention provides the composition of Wenguanguo components for modulating the release, breakdown and uptake of Acetylcholine (Ach) and its receptors in a subject. The said extracts of this invention inhibits the deep sleep created by 5HT and increase REM sleep.

This invention provides the composition of Wenguanguo components for preventing sleep paralysis in a subject.

This invention provides the composition of Wenguanguo components for increasing alertness to a sleeping subject.

This invention provides the extract or compound for regulating the activities of cell's enzymes and helping growth of the bladder and sphincter.

An immature bladder and sphincter cannot control the process and action of urination. By accelerating the growth of the bladder and the sphincter will strengthen their functions, this problem will be overcome, and enuresis will not occur.

There are many different periods of sleep a person goes through. These include Slow-Wave-Sleep 1 (SWS 1), Slow-Wave-Sleep 2 (SWS 2), Slow-Wave-Sleep 3 (SWS 3) Slow-Wave-Sleep 4 (SWS 4) and Rapid Eye Movement (REM). SWS 1 and SWS 2 are both periods of light sleep where it is relatively easy to wake someone up. Light sleep is usually more frequent in the second half of sleep. SWS 3 and SWS 4 are both periods of deep sleep, where it is difficult to wake the sleeper. Deep sleep is more frequent in the first half of sleep and each period will get shorter each time afterward. REM is a period of sleep in which people have their most vivid dreams. The wave patterns are similar to the patterns in which a person is awake. However, it is difficult to wake someone up who is in this state of sleep. The sleep cycle of a typical person can be described as follows:

SWS1, SWS2, SWS3, SWS4, SWS3, SWS2, REM, SWS1, SWS2, SWS3, SWS4, SWS3, SWS2, REM. (See FIG. 2)

However, the above sequence may not be in a fixed order. The sleep status may shift from SWS 4 to SWS1 or wake up because the body turns from one side to the other. It may shift to the SWS2 status after the movement. The interval between two REM is about 90 min. For healthy people, SWS1 will occupy about 5% of sleep, SWS2 will occupy about 50% of sleep, SWS3 will occupy about 10% of sleep, SWS4 will occupy about 10% of sleep and REM will occupy about 25% of sleep. Since a person in SWS1 and SWS2 can easily be woken, a healthy person has enough opportunities to wake up to urinate. However if a person's sleeping status is mostly in SWS4, he has less chance of waking up when the bladder is full. It is difficult for him to break through the barriers of deep sleep. Then enuresis occurs. This invention relates to a plant extract, including Wenguanguo, for preventing enuresis.

The composition comprising extracts or compounds from husks or fruit-stems or seed's shells or leaves or branches or stems or kernels or roots or barks of Wenguanguo or Xanthoceras sorbifolia can regulates the activities of cell's enzymes, strength the cell functions including growth, strengthening the urinary tract function, regulates the bone, lung, bladder, penis cell function, metallothionein gene activation, induces cells to proliferate, stimulate cells functions against diseases. This invention provides compositions comprising the active components in fractions of Xanthoceras Sorbifolia extract.

The active components of the Wanguanguo extracts stimulate the enzymes activities of bladder cell, lung cell and bone cell. Experimental results of this invention showed that lung cells began to grow at concentration of 10 ug/ml. Experimental results also showed that lung cells grew up to 120% in extract concentration of 50 ug/ml. The figure also shows the efficacy of Wanguanguo extract in growth activity of bladder cells. The bladder cells were stimulated to grow up to 125% in culture medium RPMI-1640 containing extract concentration of 10 μg/ml. It shows the efficacy of Wanguanguo extract on the activity of bone cells' growth. Bone cells grow up to 120% of controls in culture medium RPMI-1640 when used with low concentration of extract (i.e., 10 μg/ml).

The following methods and materials were used in the examples and/or experiments described below:

Cells. Human cell lines were obtained from American Type Culture Collection: HTB-9 (bladder), HeLa-S3 (cervix), DU145 (prostate), H460 (lung), MCF-7 (breast), K562 (leukocytes), HCT116 (colon), HepG2 (liver), U2OS (bone), T98G (brain) and OVCAR-3 (ovary). Cells were grown in culture medium (HeLa-S3, DU145, MCF-7, Hep-G2 and T98G in MEN (Earle's salts); HTB-9, H460, K562, OVCAR-3 in RPMI-1640; HCT-116, U2OS in McCoy-5A) supplemented with 10% fetal calf serum, glutamine and antibiotics in a 5% CO₂ humidified incubator at 37° C.

MTT Assay. The procedure for MTT assay followed the method described in (Carmichael et al., 1987) with only minor modifications. Cells were seeded into a 96-wells plate at concentrations of 10,000/well (HTB-9, HeLa, H460, HCT116, T98G, OVCAR-3), 15,000/well (DU145, MCF-7, HepG2, U2OS), or 40,000/well (K562), for 24 hours before drug-treatment. Cells were then exposed to drugs for 48 hours (72 hours for HepG2, U2OS, and 96 hours for MCF-7). After the drug-treatment, MTT (0.5 mg/ml) was added to cultures for an hour. The formation of formazan (product of the reduction of tetrazolium by viable cells) was dissolved with DMSO and the O.D. at 490 nm was measured by an ELISA reader. The MTT level of cells before drug-treatment was also measured (TO). The % cell-growth (% G) is calculated as:
% G=(TD−T0/TC−T0)×100 (1),
where TC or TD represent O.D. readings of control or drug-treated cells. When T0>TD, then the cytotoxicity (LC) expressed as % of the control is calculated as:
% LC=(TD−T0/T0)×100 (2).

Purification of the bladder stimulation components by C18 reverse-phase column chromatography with FPLC and HPLC technology.

A column with dimension of 2×30 cm containing Octadecyl-functionalized silica gel was equilibrated with 10% acetonitrile-0.005% TFA at the flow-rate of 2 ml/min. Plant extract (0.4-1 g/ml in above solution) was loaded onto the column. The bound extract was then eluted with an increasing acetonitrile gradient from 10% to 80% in a total volume of 1000 ml. The fractions were monitored at wavelength of 254 nm. Chromatography was conducted with AKTA_FPLC instrument equipped with P920 pump, UPC-900 monitor and Frac-900 fraction collector. Fractions were then collected and lyophilized for activity analysis or further purification with HPLC. HPLC was conducted with a Waters HPLC system equipped with 510 pump, automated gradient controller, 484-monitor and 754B data module recorder. A preparative column (Waters Delta Pak C18-300A) was equilibrated with 15% acetonitrile-0.005% TFA before use. A isocratic elution with 15% acetonitrile-0.005% TFA at a flow-rate of 1 ml/min. was employed to further fractionate the active growth-stimulation components. Eluted fractions were monitored at wavelength of 207 nm and 254 nm. Fractions were collected by a fraction collector. Repeated application of samples were carried out by an autosampler (DAS-10, D-Star).

This invention will be better understood from the examples which follow. However, one skilled in the art will readily appreciate that the specific methods and results discussed are merely illustrative of the invention as described more fully in the claims which follow thereafter.

### EXPERIMENTAL DETAILS

### Experiment 1

### Herb Extraction

(a) extracting Xanthoceras Sorbifolia powder of husks or branches or stems or leaves or kernels or roots or barks with organic solvent at ratio of 1:2 for 4-5 times for 20-35 hours for each time to form an organic extract; (b) collecting the organic extract; (c) refluxing the organic extract for 2-3 times at 80° C. to form second extracts; (d) removing the organic solvent from the second extract; and (e) Drying and sterilizing the extract to form a Xanthoceras Sorbifolia extract powder.

### Experiment 2

### Analysis of Xanthoceras Sorbifolia Extract Components by HPLC Chromatography

Methods and Materials

HPLC. A C-18 reverse phase μ-bondapak column (Water P/N 27324) was equilibrated with 10% acetonitrile, 0.005% Trifluoroacetic acid (equilibration solution). An extract of Xanthoceras Sorbifolia prepared as described in Experiment 1 was dissolved in equilibration solution (1 mg/ml) before being applied onto the column. 20 ug of samples was applied into column. Elution conditions: Fractions were eluted (flow rate 0.5 ml/min.) with acetonitrile (concentration gradient from 10% to 80% in 70 min) and then remains at 80% for 10 min (70-80 min). The acetonitrile then dropped to 10% (80-85 min) and remained at 10% for 25 min (85-110 mm). The fractions were monitored at 207 nm with a chart speed 0.25 cm/min and OD full scale of 0.128.

Instruments. Waters Model 510 Solvent Delivery System; Waters 484 tunable Absorbance Detector; Waters 745/745B Data Module

Absorbance analysis. The absorption profile of Xanthoceras Sorbifolia extract at various wavelengths was determined. An extract of Xanthoceras Sorbifolia prepared as described in Experiment 1 was dissolved in 10% acetonitrile/TFA and scanned at 200-700 nm with a spectrophotometer [Spectronic Ins. Model Gene Sys2].

Results

HPLC. About 60-70 peaks can be accounted for in the profile. Among them four are major peaks, 10 are medium size and the rest are small fractions. The major peaks are labeled with a to z following increased concentration of acetonitrile elution. See FIG. 1.

Absorption maximum. Three absorption maximum were identified for Xanthoceras Sorbifolia plant extract; 207 nm, 278 nm and 500 nm. See FIG. 18.

### Experiment 3

### Determination of Cell-Stimulation Component(S) of Xanthoceras Sorbifolia Extract with Cells Derived from Different Human Organs Using MTT Assay

Methods and Materials

Cells. Human cell lines were obtained from American Type Culture Collection: HTB-9 (bladder), HeLa-S3 (cervix), DU145 (prostate), H460 (lung), MCF-7 (breast), K562 (leukocytes), HCT116 (colon), HepG2 (liver), U2OS (bone), T98G (brain) and OVCAR-3 (ovary). Cells were grown in culture medium (HeLa-S3, DU145, MCF-7, Hep-G2 and T98G in MEN (Earle's salts); HTB-9, H460, K562, OVCAR-3 in RPMI-1640; HCT-116, U2OS in McCoy-5A) supplemented with 10% fetal calf serum, glutamine and antibiotics in a 5% CO₂ humidified incubator at 37° C.

MTT assay. The procedure for MTT assay followed the method described in (Carmichael et al., 1987) with only minor modifications. Cells were seeded into a 96-wells plate at concentrations of 10,000/well (HTB-9, HeLa, H460, HCT116, T98G, OVCAR-3), 15,000/well (DU145, MCF-7, HepG2, U2OS), or 40,000/well (K562), for 24 hours before drug-treatment. Cells were then exposed to drugs for 48 hours (72 hours for HepG2, U2OS, and 96 hours for MCF-7). After the drug-treatment, MTT (0.5 mg/ml) was added to cultures for an hour. The formation of formazan (product of the reduction of tetrazolium by viable cells) was dissolved with DMSO and the O.D. at 490 nm was measured by an ELISA reader [Dynatech. Model MR700]. The MTT level of cells before drug-treatment was also measured (T0). The % cell-growth (% G) is calculated as:
% G=(TD−T0/TC−T0)×100 (1)
where TC or TD represent O.D. readings of control or drug-treated cells. When T0>TD, then the cytotoxicity (LC) expressed as % of the control is calculated as:
% LC=(TD−T0/T0)×100 . (2)

Results. Among the 11 cell lines studies, their sensitivity toward Xanthoceras Sorbifolia extract can be divided into four groups (1) Growth stimulation at low concentration but inhibition at high concentration: bladder, lung and bone. (2) Strong inhibition with no growth effect: Ovary; (3) Moderate inhibition with no growth effect: leukocyte, liver, prostate, breast and brain and (4) lease sensitive or no effect: colon and cervix)

Among these cell line studied, it was found that FPLC fractions 18-23 of the Xanthoceras Sorbifolia plant extract stimulate cell growth of bladder. See FIGS. 10-11

### Experiment 4

### Purification of Active Components in the Xanthoceras Sorbifolia Extract

(A) Fractionation of Xanthoceras Sorbifolia Components with FPLC.

Methods

Column. Octadecyl functionalized silica gel; column dimension: 2 cm×30 cm; equilibrated with 10% acetonitrile—0.005% TFA.

Sample loading: 1-2 ml, concentration: 400 mg/ml in 10% acetonitrile/TFA.

Gradient elution: 10-80% acetonitrile in a total volume of 1000 ml.

Monitor absorption wavelength: at 254 nm.

Fraction Collector: 10 ml/fractions. Instrument: AKTA-FPLC, P920 pump; Monitor UPC-900; Frac-900.

Results. The elution profile shows 4-5 broad fractions. See FIG. 24. These fractions were analyzed with HPLC. FIGS. 28A, B, C and D are elution profile of fractions 17, 20, 23 and 26 respectively. Major components were identified in these fractions which include the stimulation component(s). Further purification of the stimulation component will be achieved with preparative HPLC chromatography as described in the next experiment.

Purification of Fraction #18-23 with HPLC by a Preparative C18 Column with 15% Acetonitrile Isocratic Elution

Methods

Column. A preparative column (Waters Delta Pak C18-300A) was equilibrated with 15% acetonitrile-0.005% TFA before use.

Sample loading: 50 ul, with concentration: 1-2 mg/ml.

Elution: isocratic elution with 15% acetonitrile-0.005% TFA at a flow-rate of 1 ml/min.

Monitor: Eluted fractions were monitored at wavelength of 207 nm and 254 nm.

Fraction Collector: 1 ml fraction. Instrument: HPLC was conducted with a Waters HPLC system equipped with 510 pump, automated gradient controller, 484-monitor and 754B data module recorder.

Repeated application: of samples were carried out by an autosampler (DAS-10, D-Star).

Although the present invention has been described in detail with particular reference to preferred embodiments thereof, it should be understood that the invention is capable of other different embodiments, and its details are capable of modifications in various obvious aspects. As is readily apparent to those skilled in the art, variations and modifications can be affected while remaining within the spirit and scope of the invention. Accordingly, the foregoing disclosure, description, and figures are for illustrative purpose only, and do not in any way limit the invention which is defined only by the claims.

What is claimed is:

1. A composition comprising an effective amount of extracts or compounds from husks or fruit-stems or seed's shells or leaves or branches or stems or kernels or roots or barks of Wenguanguo or Xanthoceras sorbifolia.

2. The composition of claim 1, further comprising one or any combination of the compounds showed the characteristic elution profile or peaks as shown in FIG. 1

3. The composition of claim 1, further comprising one or any combination of the compounds showed the characteristic elution profiles or peaks in the HPLC as shown in FIG. 12 A-D

4. The composition of claim 1, further comprising one or any combination of the compounds showed the characteristic elution profile or peaks as shown in FIG. 13, or FIG. 14, or FIG. 15, or FIG. 16 or FIG. 17.

5. A use of composition according to claim 1 regulates the cell's components or receptors and strength the cell functions including growth and apoptosis.

6. A use of composition according to claim 1 regulates the activities of cell's mitochondrial dehydrogenases and other enzymes.

7. A use of composition according to claim 1 for manufacture medicine or health food for curing enuresis or strengthening the urinary tract function.

8. A use of composition according to claim 1 for manufacture medicine or health food for strengthening the bladder function, urethra sphincter or the components or receptor related to them.

9. A use of composition according to claim 1 for manufacture medicament or health food for strengthening the bone cell function, lung cell function cell function or improving the response of cerebral cortex and cerebellum.

10. A use of composition according to claim 1 for manufacture medicament or health food for preventing individuals from sleeping in the extra deep level.

11. A use of composition according to claim 1 for manufacture medicament or health food for improving the sensory stretch receptor in the bladder wall to give a collect signal to central nerves system.

12. A use of composition according to claim 1 for manufacture medicament or health food for improving the nerves system.

13. A use of composition according to claim 1 for manufacture medicament or health food for stimulating cell activities against disease.

14. A use of composition according to claim 1 for manufacture medicament or health food for regulating bladder from stress, nervous, aging or hyper-reflexia, which create urgency and frequency micturition.

15. A use of composition according to claim 1 for manufacture medicament or health food for relaxing the bladder tissue which contracts due to Ach.

16. A use of composition according to claim 1 for manufacture medicament or health food for inhibiting AChE.

17. A method according to claims 1 comprising the composition for curing enuresis, frequent micturition, urinary incontinence.

18. A method according to claims 1 comprising the compositions for treatment or prevention a disease requiring enhancement of nerve growth factor production.

19. A methods according to claims 1 comprising the compositions for stimulates metallothionein gene activation

20. A method according to claims 1 comprises composition induces cells to proliferate.
